(19) United States
(12) Patent Application Publication
(10) Pub. No.: US 2003/0109802 A1
(43) Pub. Date: Jun. 12, 2003

(54) Cauterizing biopsy system

(76) Inventors: Paul F. Laeseke, Sun Prairie, WI (US); Kelly Rose Stevens, Madison, WI (US); Thomas C. Winter, Middleton, WI (US); Fred T. Lee JR., Madison, WI (US); Frank J. Fronczak, Madison, WI (US); John G. Webster, Madison, WI (US); Connie L. Davis, Mercer Island, WA (US)

Correspondence Address: QUARLES & BRADY LLP, 411 E. WISCONSIN AVENUE, SUITE 2040, MILWAUKEE, WI, 53202-4497, US

(21) Appl. No.: 10/021,622

(22) Filed: Dec. 12, 2001

(51) Int. Cl. A61B 10/00

(52) U.S. Cl. 600/564

(57) ABSTRACT

A method and apparatus for reducing bleeding in biopsy procedures provides a cauterizing electrical current through an introducer needle as the needle is withdrawn. A majority of the length of the introducer needle may be insulated to concentrate the cauterization at its tip, and a field smoothing cauterizing stylet may be used in conjunction with the tube to provide more even cauterization and/or instrumentation to monitor cauterization. Cauterization may be controlled by the speed of withdrawal of the introducer needle as guided manually or automatically by the instrumentation.

### CROSS-REFERENCE TO RELATED APPLICATIONS

### STATEMENT REGARDING FEDERALLY SPONSORED RESEARCH OR DEVELOPMENT

### BACKGROUND OF THE INVENTION

The present invention relates to biopsy systems, and in particular, to a biopsy apparatus and method providing reduced bleeding after the biopsy needle is retracted.

Biopsy is the removal of a small piece of tissue from the body in order to determine the presence of disease. A special biopsy needle may be used to penetrate the organ where the disease is suspected and to extract the tissue sample, which can then, for example, be examined under a microscope.

Current biopsy procedures may employ a special biopsy needle having a laterally opening tray into which tissue will expand. Once the needle is in position within the tissue to be sampled, the physician releases a spring-loaded sheath, which slides over the tray cutting the tissue sample free and trapping the tissue sample inside the tray. The biopsy needle is withdrawn from the patient.

When multiple biopsy samples are to be taken or when it is difficult to accurately locate the biopsy needle, a guide may be employed for the biopsy needle comprising a sharp rod (stylet) that is housed inside a hollow cylindrical tube (introducer needle). The introducer needle and stylet are inserted through the skin and into the organ of interest and then the stylet is removed from the introducer needle and replaced with the biopsy needle. The biopsy needle is longer than the introducer needle to extend outward therefrom allowing multiple samples to be taken.

The primary risk associated with liver biopsy is bleeding after the removal of the needle from the patient. Although this occurs in less than 1% of the patients, complications due to this bleeding are severe and can lead to death. One method of reducing bleeding in biopsies is described in the article Electrocautery of the Track after Needle Biopsy of the Liver to Reduce Blood Loss, by Edwin H. Kim et al., published in Investigative Radiology, Vol. 28, No. 3, pgs. 28-230 (1993).

According to this method, the outside of the biopsy needle is coated with a thin layer of electrical insulation except for the last two centimeters. A source of radio frequency electrical power is then connected to the biopsy needle as it is withdrawn from the body to provide an electrocauterization of the needle track.

A significant drawback to this approach is the need to severely limit the power of the electrocauterizing source. The investigators noted that at higher cauterizing power, there was visible thermal damage to portions of the biopsy specimen. A significant question remains as to whether biopsy specimens using even lower power are not subtly altered by this process.

A secondary risk of biopsies of cancerous tissue is the risk of the biopsy needle “seeding” cancer cells into other tissues and bloodstream as the needle is withdrawn.

### BRIEF SUMMARY OF THE INVENTION

The present invention allows the use of electrocauterization in biopsies without risk of thermal damage to the biopsy specimen. The invention thus allows the use of higher power to cauterize the track of the biopsy needle. Higher cauterizing power may provide the benefits of superior cauterization, faster procedures producing less discomfort to the patient, and has the potential of reduced risk of tumor seeding.

Generally, the invention eliminates thermal damage to the biopsy specimen by using an introducer needle as the cauterizing tool. In this way, the biopsy needle and specimen can be fully withdrawn from the patient before cauterization is initiated. Higher cauterizing power promotes greater temperature gradients in the tissue, resulting in a sharper boundary between living and cauterized tissue. Insulating the needle allows the power to be concentrated at a relatively small (few millimeter) region at the end of the introducer needle. The smaller cauterizing region may reduce charred tissue adhering to the needle such as may further promote bleeding and hinder retraction of the introducer needle.

The introducer needle may be more easily instrumented with thermocouples and the like, than the biopsy needle, to allow monitoring of the cauterizing process. The introducer needle may be further fitted with a cauterizing stylet particularly designed for cauterizing.

Thus, it is one feature of the invention that it provides for electrocauterization of a biopsy needle track without risk of heat damage to the biopsy tissue.

Another feature of the invention is that it reduces the contact area between the cauterizing probe and the tissue track. This may reduce the risk of adhesion of tissue to the cauterizing probe or tearing of tissue, and promotes higher cauterization power and greater current densities in the tissue that may produce improved and/or faster cauterization.

Yet another feature of the invention is that it allows greater control of the cauterizing probe including shaping of the cauterization area to provide more uniform cauterization currents and instrumentation of the cauterization area with temperature probes and the like.

In this respect, the invention better allows quantitative feedback with respect to the cauterization process to control the speed of withdrawal of the biopsy needle ensuring proper cauterization.

The foregoing features and advantages may not apply to all embodiments of the inventions and are not intended to define the scope of the invention, for which purpose claims are provided.

In the following description, reference is made to the accompanying drawings, which form a part of this application, and in which there is shown by way of illustration, a preferred embodiment of the invention. Such embodiment also does not define the scope of the invention and reference must be made therefore to the claims for this purpose.

### BRIEF DESCRIPTION OF THE DRAWINGS

FIG. 1 is a side elevational view of the introducer needle of the present invention showing an insulated section separating uninsulated distal and proximal ends of the needle, the former of which is connected to a cauterizing electrical source;

FIG. 2 is a fragmentary cross sectional view of the proximal end of the needle of FIG. 1 when fitted with an introducer stylet for insertion into the patient;

FIG. 3 is a figure similar to that of FIG. 2 showing the introduction of a biopsy needle into the introducer needle of FIG. 1 after removal of the introducer stylet and with the tray of the biopsy needle open for receiving biopsy specimens;

FIG. 4 is a figure similar to that of FIG. 4 showing extension of a biopsy needle sheath to cut and trap tissue within the tray of the biopsy needle;

FIG. 5 is a figure similar to that of FIG. 2 showing the introducer needle of FIG. 1 after the biopsy needle and introducer stylet are removed, during withdrawal of the introducer needle as cauterizing electrical power is applied;

FIG. 6 is a figure similar to that of FIG. 5 showing an alternative embodiment of the invention in which the cauterizing electrical power is applied through a specially shaped cauterizing stylet inserted into the introducer needle and showing two alternative locations of thermocouple placement;

FIG. 7 is a schematic representation of the introducer needle after insertion into the patient as connected to an external cauterizing power supply and monitoring equipment;

FIG. 8 is a fragmentary perspective view of the distal end of the introducer needle of FIG. 1 showing an alternative embodiment having a combination power clamp and display, the latter providing guidance for how fast to withdraw the introducer needle; and

FIG. 9 is a simplified schematic diagram of a mechanism to provide for automated withdrawal of the introducer needle using feedback signals obtained from the introducer needle during the cauterization.

### DETAILED DESCRIPTION OF THE PREFERRED EMBODIMENT

Referring now to FIG. 1, the biopsy system 10 of the present invention includes an introducer needle 12 comprised of a tube 14 of an electrically conductive biocompatible metal, for example, stainless steel. In an example embodiment, the tube 14 may have an outer diameter of approximately 1.2 mm. and be 15 centimeters in length. Introducer needles having tubes 14 of this type are well known in the art and commercially available from a number of sources.

The introducer needle 12 of the present invention differs from those that are commercially available by the application of an outer electrically insulating coating 30 that produces a circumferentially continuous insulated section 18 extending along the tube 14 less than the fall length of the tube 14 so as to expose a proximal end 20 of the tube 14 and a distal end 22 of the tube 14. The exposed portion of the proximal end 20 in a preferred embodiment extends 4 mm.

The insulated section 18 may comprise any biocompatible insulator, however, the present invention uses a layer of Parylene C vapor deposited to a thickness of approximately 0.001 inches. Parylene C is commercially available from the Vitek Research Corporation of Derby, Connecticut and has a lubricity approaching that of Teflon to present a coefficient of friction approximately equal to 0.29. Other insulating materials may also be used.

The distal end 22 of the tube 14 is partially received by a handle 24 that may be grasped during insertion or removal of the introducer needle 12 into or out of the patient. The handle 24 is of electrically insulating material such as a moldable thermoplastic. An exposed portion of the distal end 22 before the handle 24 may accept an electrical clamp 26 to conduct radio frequency electrical power through the tube 14 beneath the insulating section 18 to the proximal end 20 as will be described.

Printed on or visible through the insulated section 18 are graduation marks 28 allowing the physician to determine the insertion depth of the introducer needle 12. A proximal zone 31 of distinct marking or color preceding the proximal end 20 is provided to indicate to the physician that the proximal end 20 is about to pass out of the patient so that the radio frequency electrical power may be turned off.

Referring now to FIG. 2, before the biopsy, a rod-shaped stylet 32 is fit snugly within the bore of the tube 14 having a sharpened end protruding from the proximal end 20 of the tube 14 to ease the insertion of the introducer needle 12 into the body tissue 34. The proximal end 20 of the introducer needle 12 is positioned within tissue 34 near the site where a biopsy will be taken using ultrasound or radiographic imaging.

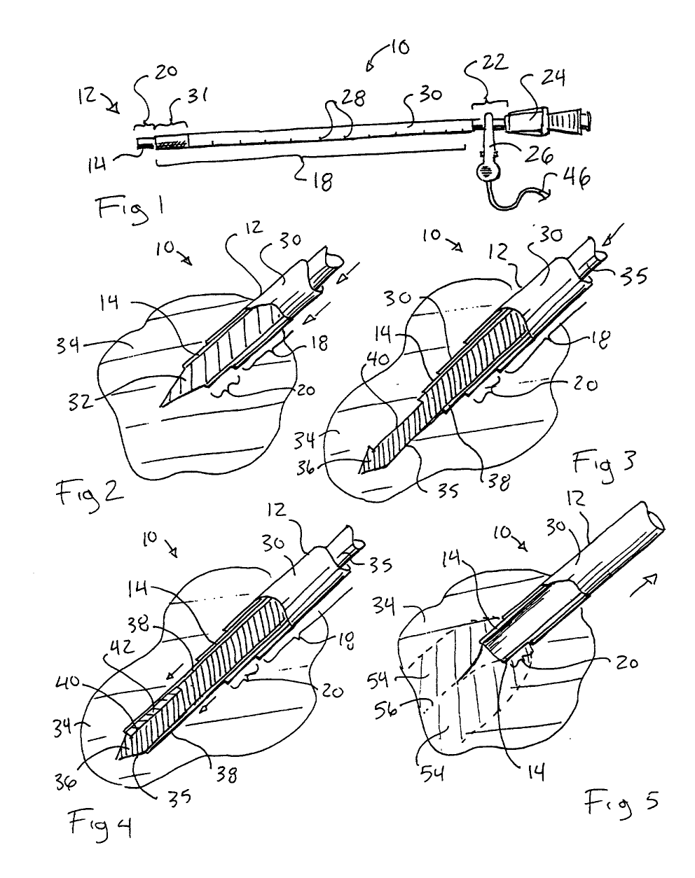

Referring to FIG. 3, after the introducer needle 12 is correctly positioned, the stylet 32 of FIG. 2 is removed and replaced with a biopsy needle 35 providing a central shaft 36 and an outer concentric tubular sheath 38 drawn back from the proximal end to expose a laterally opening tray 40 in the shaft 36. The proximal end of the central shaft 36 of the biopsy needle 35 is sharpened like the stylet 32 allowing the biopsy needle 35 to extend easily beyond the proximal end 20 of the tube 14 so that tissue 34 may enter the tray 40. Biopsy needles suitable for use with the present invention are commercially available from a number of sources including Cook Urological of Spenser, Ind., and C. R. Bard, Inc. of Covington, Ga.

Referring now to FIG. 4, once the central shaft 36 of the biopsy needle 35 is in place, the sheath 38 may be driven forward by a spring mechanism over the tissue 42 within the tray 40 to sever the tissue 42 from the remaining tissue 34 and hold it within the tray 40.

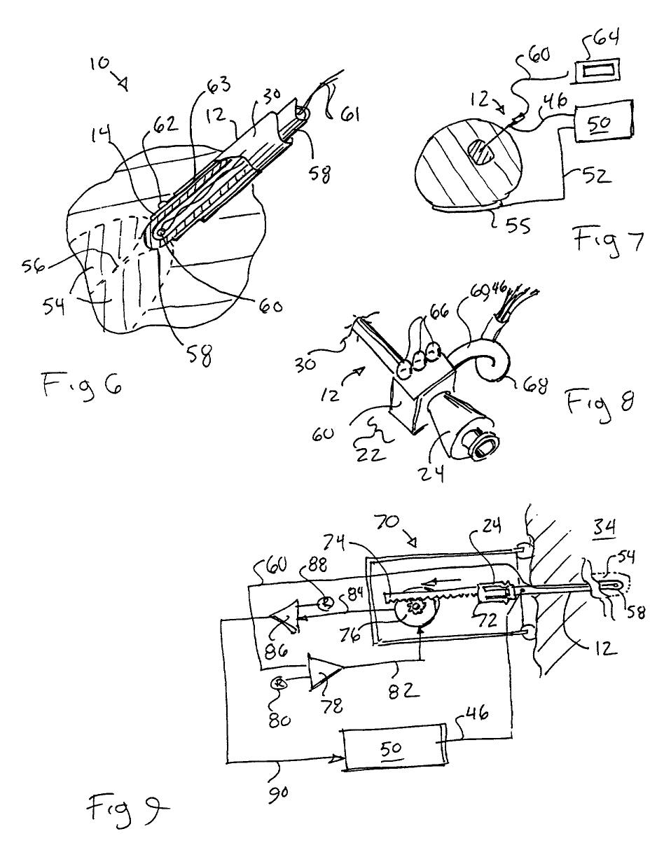

Referring now to FIG. 5, the biopsy needle 35 is now withdrawn and the biopsy tissue 42 removed. The biopsy needle 35 may be reinserted into the introducer needle 12 and additional biopsy samples taken. Upon conclusion of the sampling as shown in FIG. 5, the biopsy needle 35 is removed leaving only the introducer needle 12.

Referring now to FIGS. 1, 5 and 7, at this time, the clamp 26 shown in FIG. 1 may be attached to the distal end 22 of the introducer tube to provide, via a power lead 46, a connection to a radio frequency electrical power source 50. This power source may be a standard cauterizing electrical generator such as the RITA Model 1500 RF Generator available from Rita Medical Systems, Inc. of Mountain View, Calif. whose specifications include an adjustable power of from 0 to 150 watts at a frequency of 460 kHz. A return lead 52 of the power source 50 is connected to a large area conducted pad 55 of a type well known in the art to allow for complete circuit through the patient without the concentration of current flow that would produce cauterization temperatures occurring anywhere except at the introducer needle 12.

Referring still to FIG. 5, as cauterizing electrical power is applied to the tube 14, it is transferred to the tissue 34 through the exposed conductive surface of the proximal end 20 of the tube 14. For a moment after connection of the cauterizing electrical power to the introducer needle 12, the introducer needle is held in place to allow the adjacent tissue 34 to rise to cauterizing temperatures. Then the introducer needle 12 is withdrawn, producing a cauterization region 54 along the biopsy track 56. In the preferred embodiment, a wattage setting of between 100 and 150 watts may be used. While the Applicants do not wish to be bound by a particular theory, it is believed that higher wattages focused in the limited area of the proximal end 20 produce higher current densities and greater temperature gradients resulting in a sharper boundary between the cauterization region 54 and living tissue 34.

Referring now to FIG. 6, in an alternative embodiment, an electrically conductive cauterizing stylet 58 may be introduced into the introducer needle 12 before the cauterization procedure. The cauterizing stylet 58 may be connected directly to the cauterizing power source in lieu of a connection to the distal end 22 of the tube 14, or may receive electrical power through its intimate contact with the interior conductive portion of the tube 14. In an alternative embodiment, the insulation of the tube 14 may be eliminated or supplemented with an insulating outer coating 63 positioned between the tube 14 and the cauterizing stylet 58.

The proximal end of the cauterizing stylet 58 is rounded to provide more even field lines than those produced by the sharp edges of the tube 14, limiting hot spots that may lead to uneven cauterization or burning.

The cauterizing stylet 58 may include an internal thermocouple 60 so as to allow monitoring of the temperature at the cauterizing region with instrumentation leads 61 passing through a central bore of the cauterizing stylet 58. Alternatively, a thermocouple 62 may be placed directly on the proximal end 20 of the introducer needle 12 to measure temperature at this location, with instrumentation leads 61 passing between the insulating material 30 and the outer wall of tube 14. In yet a further embodiment, a thermocouple is placed down the middle of the introducer needle 12 without the cauterizing stylet 58. A suitable thermocouple is the Endocare CRYOcare™ thermocouple commercially available from Endocare, Inc. of Irvine, Calif.

Referring again to FIG. 7, the thermocouple 60 or 62 may communicate via instrumentation leads 61 to instrument readout 64 providing, for example, an LCD display of the cauterizing temperature or a synthesized voice temperature readout. Ideally, the temperature will be monitored to be within 65 to 80 degrees Celsius. Alternatively, the read out may indicate a desired speed of retraction of the introducer needle 12 based on these temperature ranges. As mentioned above, the present inventors have determined that before withdrawal of the introducer needle 12, a cauterizing holding time should be observed until the temperature rises to within this range. At that time, the introducer needle 12 is retracted at a speed adjusted to maintain a cauterizing temperature within this range. In an alternative embodiment, power dissipation or electrical resistance may be measured to guide in the cauterization process.

Referring now to FIG. 8, in order to provide the attending physician with guidance as to the retraction speed of the introducer needle 12, the instrument readout 64 may be moved to the distal end 22 of the introducer needle 12 to provide a display thereupon. For example, the display may provide for three light emitting diodes (LEDs) 66 of different colors: a red LED lighting to indicate a temperature below 65 Celsius, a yellow LED to indicate a temperature between 65 and 80 degrees Celsius and a green LED indicating a temperature above 80 degrees Celsius. The colors also indicating generally to the physician how speed of retraction of the introducer needle 12 should be adjusted with red indicating a slowing or stopping of the retraction; green indicating a speeding or starting of the retraction; and yellow indicating that the proper retraction speed has been obtained.

The instrument readout 64 may also provide the connection between the power lead 46 and the tube 14 (not visible in FIG. 8), replacing electrical clamp 26 shown in FIG. 1. A unified cable 68 may be provided carrying both the power lead 46 of FIG. 7 and the instrumentation leads 61 to the radio frequency electrical power source 50 and instrument readout circuitry driving the LEDs 66 according to techniques well known in the art.

Referring now to FIG. 9, the ability to monitor the temperature or other properties of the cauterization region 54 raises the possibility of automatic retraction of the introducer needle 12 during the cauterization procedure using an automated retracting device 70. In one embodiment of such a device, the retracting device 70 may include a collet 72 receiving the handle 24 of the introducer needle 12. The collet may attach to a motor driven retracting rack 74 controlled by motor 76 such as a DC servo or stepper motor. Thermocouple signals through instrumentation leads 61 may be provided to a servo amplifier 78 operating through well known techniques to receive a reference temperature 80 to provide a control signal 82 to the motor adjusting its speed according to the thermocouple measurement. In this case, low temperatures cause a slowing of the motor; higher temperatures cause a speeding or stopping of the motor.

The motor 76 may also provide a position output signal 84 to be received by a comparator 86 detecting, by means of a reference voltage 88, that the proximal end 20 of the introducer needle 12 is about to pass out of the skin and providing a disable signal 88 to the power source 50 to stop power at this time. This corresponds to the physician ceasing radio frequency electrical power when the proximal zone 31 shown in FIG. 1 of the introducer needle 12 is exposed. In practice, the retracting device 70 might be attached to the handle 24 only upon completion of the biopsy for automated withdrawal of the retracting.

Preliminary use of this device indicates that an average time of withdrawal from 10 to 15 seconds provides an even cauterization.

It is specifically intended that the present invention not be limited to the embodiments and illustrations contained herein, but that modified forms of those embodiments including portions of the embodiments and combinations of elements of different embodiments also be included as come within the scope of the following claims.

We claim:

1. A biopsy needle assembly comprising:
an introducer shaft having a first and second end, and sized for percutaneous insertion into a patient along an insertion path to locate the first end at a biopsy site, the first end supporting an electrically conductive surface exposed to tissue and communicating by means of an insulating conductor to the second end to connect with a cauterizing electrical source; and
a biopsy needle interfitting with the introducer shaft to be guided thereby, the biopsy needle including a sampling means for removal of a tissue sample before cauterization of the insertion path using the electrically conductive surface.

2. The biopsy needle assembly of claim 1 wherein the introducer shaft is a hollow tube and the biopsy needle slides within the hollow tube.

3. The biopsy needle assembly of claim 1 wherein the introducer shaft is electrically conductive and the electrically conductive surface is a first end of the introducer shaft.

4. The biopsy needle assembly of claim 3 wherein the insulated conductor is a middle portion of the introducer shaft having an outer insulating covering.

5. The biopsy needle assembly of claim 1 wherein the electrically conductive surface is a conductive stylet having a first end supported by the introducer shaft.

6. The biopsy needle assembly of claim 1 wherein the conductive stylet has a rounded tip.

7. The biopsy needle assembly of claim 5 wherein the introducer shaft is a hollow tube having an outer conductive covering and wherein the insulated conductor is provided by a portion of the conductive stylet fitting within the hollow tube.

8. The biopsy needle assembly of claim 5 wherein a shaft portion of the conductive stylet includes an outer insulating covering to provide the insulated conductor.

9. The biopsy needle assembly of claim 1 wherein the electrically conductive surface extends continuously by less than 2 cm as measured along the insertion path.

10. The biopsy needle assembly of claim 1 further including a temperature sensor positioned at the electrically conductive surface.

11. The biopsy needle assembly of claim 1 further including indicia on the introducer shaft near the first end to indicate to a user that the electrically conductive surface is about to be withdrawn through the skin.

12. The biopsy needle assembly of claim 1 further including an indicator providing guidance to a user as to a speed of withdrawal of the introducer shaft during cauterization.

13. The biopsy needle assembly of claim 12 wherein the indicator is selected from the group consisting of:
(a) a visual display providing an indication of at least one of temperature, electrical resistance, and electrical power at the electrically conductive surface;
(b) an audio signal providing an indication of at least one of temperature, electrical resistance, and electrical power at the electrically conductive surface;
(c) a visual display providing an indication of changes in a desired speed of retraction of the introducer needle based on at least one of temperature, electrical resistance and electrical power at the electrically conductive surface; and
(d) an audio signal providing an indication of changes in a desired speed of retraction of the introducer needle based on at least one of a temperature, electrical resistance and electrical power at the electrically conductive surface.

14. The biopsy needle assembly of claim 1 further including a retracting mechanism for automatically removing the introducer shaft according to a predetermined schedule.

15. The biopsy needle assembly of claim 13 wherein the retracting mechanism controls the schedule for retracting the introducer shaft according to at least one of a temperature, electrical resistance and electrical power at the electrically conductive surface.

16. A method of performing a biopsy comprising:
(a) inserting an introducer shaft percutaneously into a patient along an insertion path to locate a first end of the introducer shaft at a biopsy site, the first end supporting an electrically conductive surface exposed to tissue;
(b) guiding a biopsy needle with the introducer shaft to the biopsy site to obtain a tissue sample from the biopsy site;
(c) removing the biopsy sample from the patient; and
(d) connecting a cauterizing electrical source to the electrically conductive surface to cauterize tissue along the insertion path.

17. The method of claim 16 wherein the electrically conductive surface contacts tissue at only a portion of the insertion path and including the step of withdrawing the introducer shaft while the cauterizing electrical source is connected to the electrically conductive surface.

18. The method of claim 17 including the step of disconnecting the cauterizing electrical power from the electrically conductive surface prior to withdrawal of the electrically conductive surface through the skin.

19. The method of claim 16 including the step of measuring a parameter selected from the group consisting of temperature at the electrically conductive surface, resistance at the electrically conductive surface, and power deposited at the electrically conductive surface.

20. The method of claim 16 including the step of adjusting the retraction of the introducer needle during cauterization according to the measured parameter.
